(19) United States
(12) Patent Application Publication
(10) Pub. No.: US 2013/0177904 A1
(43) Pub. Date: Jul. 11, 2013

(54) ARNT ISOFORM 3 AS A PREDICTOR OF AMINOFLAVONE RESPONSIVENESS IN CANCER CELLS

(71) Applicants: Averell L. Gnatt, Pikesville, MD (US); Edward A. Sausville, Silver Spring, MD (US); Phillip Shelton, Baltimore, MD (US); Hui Yang, Catonsville, MD (US)

(72) Inventors: Averell L. Gnatt, Pikesville, MD (US); Edward A. Sausville, Silver Spring, MD (US); Phillip Shelton, Baltimore, MD (US); Hui Yang, Catonsville, MD (US)

(73) Assignee: UNIVERSITY OF MARYLAND, BALTIMORE, Baltimore, MD (US)

(21) Appl. No.: 13/580,563

(22) PCT Filed: Feb. 23, 2011
(86) PCT No.: PCT/US2011/025856

§ 371 Date: Aug. 22, 2012

Related U.S. Application Data
(60) Provisional application No. 61/307,479, filed on Feb. 24, 2010.

(51) Int. Cl. C12Q 1/68; G01N 33/68

(52) U.S. Cl. CPC C12Q 1/6886; G01N 33/6872
USPC 435/6.11; 435/7.1

(57) ABSTRACT

The present invention is directed to methods for determining whether a selected cancer is susceptible to an activity of an arylhydrocarbon receptor agonist, such as aminoflavone, via screening the cancer for expression of isoform 3 of aryl hydrocarbon nuclear translocator.

### FIELD

The invention generally relates to cancer treatment and to a method for using a specific biomarker, aryl hydrocarbon nuclear translocator isoform 3, as a predictor for sensitivity of cancer cells to treatment with an arylhydrocarbon receptor agonist, such as aminoflavone.

### BACKGROUND

Breast cancer is the second most common type of cancer afflicting women, with one in eight women estimated to be diagnosed with breast cancer in their lifetime (Jemal, A., et al., CA Cancer J Clin 59(4):225-49 (2009)). Despite improvements in current therapies, resistance ultimately emerges and it is therefore essential to develop novel strategies for the effective treatment of breast cancer.

Flavonoids, both natural and synthetic, have been recognized as exhibiting various biological activities including inhibition of protein kinase C, aromatase, and topisomerase, and as having cyclin-dependent kinase activities. In particular, 5,4′-diaminoflavones reportedly exhibit cytotoxicity against, for example, the human breast cancer cell line MCF-7 (Akama et al., J Med Chem 41:2056-2067 (1998)). In a large-scale, anti-tumor drug screen involving sixty cell lines (NCI 60-cell line panel) performed by the National Cancer Institute (NCI), aminoflavone (AF; 5-amino-2-(4-amino-3-fluorophenyl)-6,8-difluoro-7-methylchromen-4-one; NSC 686288) and other substituted flavone agonists of the arylhydrocarbon receptor were shown to have anti-tumor activity towards selected breast, renal, and ovarian cancers ((Kuffel, M. J., et al., Mol Pharmacol 62(1):143-53 (2002); Akama, T., et al., J Med Chem 40(12):1894-900 (1997); Akama, T., et al., J Med Chem 39(18):3461-9 (1996); Loaiza-Perez, A. I., et al., Mol Cancer Ther 3(6):715-25 (2004); Bengal, E., et al., Mol Cell Biol 11(3):1195-206 (1991); Monks A, et al., Anticancer Drug Des 12:533-541 (1997)). AF also proved very active in estrogen receptor α-positive (ER+) breast cancer cells, with estrogen receptor negative (ER−) breast cancer cells non-responsive (Akama, T., et al., J Med Chem 39(18):3461-9 (1996); Holbeck, S. L., Eur J Cancer 40(6):785-93 (2004)). In vivo effects of AF were evaluated employing breast cancer MCF-7 xenografts, and compatible anti-proliferative results for both in vitro and in vivo studies led to entry of AF into clinical trials (Loaiza-Perez, A. I., et al., Mol Cancer Ther 3(6):715-25 (2004)).

AF and other hydrocarbons activate the arylhydrocarbon receptor (AhR). The AhR is normally found in an inactive form as a cytosolic transcription factor bound to several chaperone proteins, which include Hsp90, prostaglandin E synthase 3, and AIP (for a review see Beischlag, T. V., et al., Crit Rev Eukaryot Gene Expr 18(3):207-50 (2008)). Upon binding of cognate ligands, classically dioxin and many similar hydrophobic moieties, the AhR is translocated to the nucleus where the receptor disassociates from its chaperone proteins and dimerizes with the aryl hydrocarbon nuclear translocator (ARNT). In the nucleus, the AhR-ARNT complex acts as a transcription factor binding to xenobiotic response elements (XRE) located on promoters governing the transcription of genes causing xenobiotic metabolism (Ikuta, T., et al., J Biol Chem 273(5):2895-904 (1988); Ikuta, T., et al., J Biochem 127(3):503-9 (2000); Kazlauskas, A., et al., Mol Cell Biol 21(7):2594-607 (2001); Whitlock, J. P., Annu Rev Pharmacol Toxicol 39:103-25 (1999)). In the case of AF, CYP1A1 protein initiates the conversion of AF into a series of active metabolites, which form covalent adducts with RNA and DNA, causing oxidative damage to DNA and DNA double stranded breaks and eventually, apoptosis (Kuffel, M. J., et al., Mol Pharmacol 62(1):143-53 (2002); Loaiza-Perez, A. I., et al., Mol Cancer Ther 3(6):715-25 (2004); Meng, L. H., et al., Cancer Res 65(12):5337-43 (2005); McLean, L., et al., Int J Cancer 122(7):1665-74 (2008); Meng, L. H., Oncogene 26(33):4806-16 (2007); Pobst, L. J. and M. M. Ames, Cancer Chemother Pharmacol 57(5):569-76 (2006); Meng, L. H., et al., J Pharmacol Exp Ther 325(2):674-80 (2008); Zacharewski, T. R., et al., Cancer Res 54(10):2707-13 (1994)). As a result of double strand DNA breaks, AF treatment of sensitive cells results in phosphorylation of H2AX, a histone 2A variant, which is phosphorylated in response to DNA damage (Pobst, L. J. and M. M. Ames, Cancer Chemother Pharmacol 57(5):569-76 (2006)) and also in the phosphorylation of pro-apoptotic p53, which stabilizes its activity. In turn, p53 downstream gene targets p21^{Wafl/Cipl} and MDM2 are activated (Kuffel, M. J., et al., Mol Pharmacol 62(1):143-53 (2002); Meng, L. H., et al., Cancer Res 65(12):5337-43 (2005); McLean, L., et al., Int J Cancer 122(7):1665-74 (2008); Meng, L. H., Oncogene 26(33):4806-16 (2007)).

As indicated here, flavonoids, such as aminoflavone (AF), and other AhR agonists have the potential to be potent anti-tumor agents. However, their activity is limited to susceptible types of cancer. An advance indication as to whether a particular cancer is likely to be susceptible to the effects of a particular drug could greatly aid in the effective and efficient treatment of cancer. There is an unmet need for the identification of biomarkers that are correlated with the susceptibility of a particular cancer to an AhR agonist, such as a flavonoid, and that can thus be surveyed as a part of the decision-making process when appropriate treatments for particular cancers are being determined.

### SUMMARY

Through diligent efforts it has been found that the presence of isoform 3 of the aryl hydrocarbon nuclear translocator (ARNTiso3) correlates with an increased sensitivity in cancer cells to arylhydrocarbon receptor (AhR) agonists, including flavonoids, such as aminoflavone. ARNTiso3 can therefore serve as a biomarker for the potential effectiveness of an AhR agonist, including a flavonoid, such as aminoflavone, against cancer, such as breast cancer.

In a first aspect, provided herein are methods for determining whether a selected cancer is susceptible to an activity of an AhR agonist, comprising screening the cancer for expression of an isoform of aryl hydrocarbon nuclear translocator (ARNT).

In a second aspect, provided herein are methods for determining whether a selected cancer is susceptible to an activity of an AhR agonist, comprising screening the cancer for expression of isoform 3 of ARNT.

In a third aspect, provided herein are methods for determining whether treatment of a subject having cancer with an AhR agonist will be effective, comprising screening the cancer for expression of an isoform of ARNT.

In a fourth aspect, provided herein are methods for determining whether treatment of a subject having cancer with an AhR agonist will be effective, comprising screening the cancer for expression of isoform 3 of ARNT.

In the third and fourth aspects, the determining is conducted before or after the subject begins said treatment.

In a fifth aspect, provided herein are methods for screening a subject having cancer for sensitivity to treatment with an AhR agonist, comprising assaying a biological sample obtained from the subject for expression of an isoform of ARNT.

In a sixth aspect, provided herein are methods for screening a subject having cancer for sensitivity to treatment with an AhR agonist, comprising assaying a biological sample obtained from the subject for expression of isoform 3 of ARNT.

In preferred embodiments of the fifth or sixth aspects, the biological sample is a tissue biopsy.

In preferred embodiments of each of these aspects, the AhR agonist is aminoflavone (AF) or a derivative thereof.

In preferred embodiments of each of these aspects, the screening or assaying is via polymerase chain reaction (PCR) or an immunoassay. In one embodiment, the immunoassay is performed using an anti-ARNTiso3 specific antibody.

In preferred embodiments of each of these aspects, the cancer is breast cancer.

In a seventh aspect, provided herein is an antibody that specifically binds ARNT isoform 3. In the seventh aspect, the antibody is a monoclonal antibody or a polyclonal antibody.

### BRIEF DESCRIPTION OF THE DRAWINGS

FIG. 1. AF reduces both ER+ and ER− breast cancer cell proliferation. The MTS cell proliferation assay was performed for the listed cell lines as suggested by the manufacturer (MTS assay, Promega). The values of increasing concentrations of AF and percent proliferation relative to vehicle treated cells are shown as a semi-log plot. Cells were assayed five days after treatment. IC50 values were estimated directly from the graph and conform to model IC50 values. Standard error of the mean was generally small and was plotted along with the mean.

FIG. 2. AF induced apoptosis is associated with DNA breaks, but not necessarily caspase 3/7 activation. 100 nM AF was employed in experimental procedures. FIG. 2A: Relative apoptosis was measured employing the Cell Death Detection ELISA^{PLUS} Assay (Roche) as described by the manufacturer. Control cells were treated with vehicle alone (Control). Induction of apoptosis was determined 24 and 48 hours post-AF treatment. At 48 hours, prominent induction of apoptosis is observed for MCF7, T47D and MDA-MBA-468 cell lines. FIG. 2B depicts results of a Western analysis, which detects gamma-H2AX, an indicator of double stranded breaks and beta-actin as control. Controls include untreated cells (−AF). Since MDA-MBA-231 is not AF responsive, it serves as a control for the AF responsive cell lines. Signals for gamma-H2AX are apparent only for AF sensitive cells. In FIG. 2C, enzymatic activity indicated relative to vehicle treated cells alone, of the sum of Caspases 3 and 7 is presented. T47D and MDA-MB-468 display Caspases 3 and 7 activity, whereas MCF7 and MDA-MB-231 do not.

FIG. 3. ChIP data reveals transcriptional crosstalk between the AhR and ER upon AF induction. Standard ChIP analysis was performed employing the Chromatin Immunoprecipitation (ChIP) Assay Kit (Millipore) as per company recommendations. For all panels shown, PCR fragments were separated on a 2% agarose gel, and stained with Ethidium Bromide. “M” represents DNA markers, and “NC” a negative control using beads without antibody. “Input” represents PCR products prior to immunoprecipitation, and is indicative of the total amount of the specific DNA prior to specific precipitation by antibodies. FIGS. 3A-D: the cell line employed (MCF7 or T47D) and the promotor identified by the PCR products (CYP1A1 or PS2) are listed above the image. Antibodies employed for precipitation are listed between panels. DNA was extracted from cells before adding AF, or 4 or 8 hours post-treatment with 100 nM AF. FIG. 3E: for control, primers, which enable amplification of a 174-bp fragment genomic DNA between the GAPDH gene and the CNAP1 gene were employed to detect nonspecific DNA prior to (input), and post (ChIP) precipitation with antibodies as indicate above the gel. The two gels represent MCF7 and T47D respectively.

FIG. 4. PCR detection of ARNTiso3. FIG. 4A: cDNA derived from the listed breast cancer cell lines were subjected to a 3% Nusieve Agarose gel (FMC) electrophoresis and stained with ethidium bromide. PCR employed the ARNT forward primer 5′ACTGCCAACCCCGAAATGAC3′ and the reverse primer: 5′CCGCCGTTCAATTTCACTGT3′. FIG. 4B: detection of ARNTiso3 as a sole product. A sole 155 base pair ARNTiso3 band is observed. Negative controls employed samples lacking DNA to rule out contamination. “M” represents marker bands of 100 and 200 bp. The assay employs a splice specific reverse primer, which overlaps exon 5, and is not found in ARNT isoforms 1 and 2 so that the reverse primer is unique for ARNTiso3 alone. Though signals of the splice-site specific PCR products are not very strong, they are visible only in the AF sensitive cell lines tested.

FIG. 5. RT-PCR indicates varied ARNTiso3 expression in different cancer cell lines from different tissues. RNA derived from the 60-cancer cell lines, was kindly provided by the NCI DTP program. RT-PCR fragments were separated on a 1% Nuseive agarose gel, and stained with Ethidium Bromide. For all panels, the left-most lane contains a DNA marker, and the right-most lane represents a reaction without cDNA template as controls. For each panel, the source of the tissue is presented on the right of the image and specific cell lines are listed above each lane respectively. As a further control, RNA was extracted from MCF7 and MDA-MB-231 cells and the same PCR reactions were performed, where MCF7 exhibited ARNTiso3 and MDA-MB-231 lacked ARNTiso3. These two cell lines also appear in Panel A, the breast cancer panel where the left side contains PCR fragments of the NCI-DTP origin and the left most samples are those originating in house.

FIG. 6. Spot-check verification of ARNTiso3 mRNA detection in RNA samples from the NCI-DTP 60-cell line panel. A “spot-check” of most of the NCI-DTP RNA samples was performed by RT-PCR as described in the Examples and FIGS. 4 and 5 with different primers. The forward primer was 5′-ATGTACCATCACTGGGTCCA-3′ and the reverse primer was 5′-TGATGTAGGCTGTCATCTTG-3′. The data generally conforms to that of FIGS. 4 and 5. On the right of each image a DNA marker is found, and on the two left-most lanes, MCF7 and MDA-MB-231 samples for control.

### DETAILED DESCRIPTION

Provided herein are novel screening methods based on the discovery of a correlation between the expression of isoform 3 of the aryl hydrocarbon nuclear translocator (ARNTiso3) by cancer cells and susceptibility or sensitivity of the cancer cells to an AhR agonist, including a flavonoid, such as aminoflavone (AF). In particular, a strong correlation between expression of ARNTiso3 by breast cancer cells and sensitivity of the cells to AF has been established. ARNTiso3 can thus act as a predictive biomarker for the sensitivity of cancer cells to treatment with an AhR agonist, including a flavonoid such as AF or a derivative thereof.

The methods of the present invention include methods for determining whether a selected cancer is susceptible to an activity of an AhR agonist, comprising screening the cancer for expression of an isoform of ARNT, such as ARNTiso3.

The methods of the present invention also include methods for determining whether treatment of a subject having cancer with an AhR agonist will be effective, comprising screening the cancer for expression of an isoform of ARNT, such as ARNTiso3.

The methods of the present invention further include methods for screening a subject having cancer for sensitivity to treatment with an AhR agonist, comprising assaying a biological sample obtained from the subject for expression of an isoform of ARNT, such as ARNTiso3.

An antibody that specifically binds ARNTiso3 is also encompassed within the scope of the invention.

### Methods of Screening and Assaying for Expression of an Isoform of ARNT

The screening methods that form the basis of the present invention are based on the detection of an expression product of a gene coding for a particular isoform of ARNT, such mRNA or the protein itself, in a biological sample. Expression of ARNTiso3 by a particular cancer indicates that the cancer will be susceptible to the effects of an AhR agonist, including AF. The screening methods are therefore only limited in their ability to determine whether particular isoforms of ARNT are being expressed by the cancer.

The screening methods begin with the collection of a biological sample from a subject having cancer or being suspected of having a cancer. The particular screening method will govern the suitability of the form and source of the biological sample, but a tissue biopsy of a tumor or lesion from the subject will generally be an excellent biological sample. The term “biological sample” generally refers to a sample obtained from a subject having cancer or that is suspected of having cancer. The source and form of the biological sample is only limited in that it contain a detectable amount of the nucleic acid sequence (e.g., DNA or mRNA) or amino acid sequence (e.g., protein) for which the sample is being assayed. Suitable examples include a tissue sample (e.g., a biopsy, a normal or benign tissue sample, a metastatic sample) and a body fluid sample (e.g., any body fluid in which cancer cells or acellular nucleic acid may be present, including, without limitation, blood, bone marrow, cerebral spinal fluid, peritoneal fluid, pleural fluid, lymph fluid, ascites fluid, serous fluid, sputum, lacrimal fluid, stool, and urine). Tissue samples and body fluids can be readily collected using any of the methods well known in the art.

To measure mRNA levels, cells in a biological sample can be lysed by techniques known to the skilled artisan and the mRNA levels in the lysates can be quantified by any of the many methods known the art. Such methods include, without limitation, hybridization assays using detectably-labeled, gene-specific DNA or RNA probes, and quantitative or semi-quantitative PCR (polymerase chain reaction) methodologies using appropriate gene-specific oligonucleotide primers. Alternatively, quantitative or semi-quantitative in situ hybridization assays can be performed using, for example, unlysed tissues or cell suspensions, and detectably (e.g., fluorescently- or enzyme-) labeled DNA or RNA probes. Additional methods for quantifying mRNA levels include RNA protection assays (RPA), cDNA and oligonucleotide microarrays, and colorimetric probe based assays.

As described in the Examples, an exemplary method of screening is through the use PCR whereby the biological sample is screened for expression of a gene encoding an isoform of ARNT, such as ARNTiso3. Nucleic acid is extracted from the biological sample using standard extraction methods known in the art and amplified using PCR for detection.

The PCR technique is well known in the art. For a review of PCR methods and protocols see, e.g., Innis et al. eds. PCR Protocols. A Guide to Methods and Application, Academic Press, Inc., San Diego, Calif., 1990. PCR reagents and protocols are also available from commercial vendors, such as Roche Molecular Systems. In the present invention, the initial template for primer extension is typically first strand cDNA that has been transcribed from RNA. Reverse transcriptases suitable for synthesizing a cDNA from the RNA template are well known. PCR is most usually carried out as an automated process with a thermostable enzyme. In this process, the temperature of the reaction mixture is cycled through a denaturing region, a primer annealing region, and an extension reaction region automatically. Sequence-specific probe hybridization is a well known method of detecting desired nucleic acids in a sample comprising cells, tissues, biological fluids and the like. Under sufficiently stringent hybridization conditions, the probes hybridize specifically only to substantially complementary sequences. The stringency of the hybridization conditions can be relaxed to tolerate varying amounts of sequence mismatch. If the target is amplified, detection of the amplified product utilizes this sequence-specific hybridization to insure detection of only the corrected amplified target, thereby decreasing the chance of a false positive. A number of hybridization formats are well known in the art including but not limited to solution phase, solid phase, mixed phase, or in situ hybridization assays. Techniques such as real-time PCR systems have also been developed that permit analysis, e.g, quantification of amplified products during a PCR reaction. The hybridization complexes are detected according to well known techniques and are not a critical aspect of the present invention. Nucleic acid probes capable of specifically hybridizing to a target can be labeled by any one of several methods typically used to detect the presence of hybridized nucleic acids.

For polymerase chain reaction (PCR), an annealing temperature of about 5° C. below Tm is typical in stringent amplification, although annealing temperatures vary between about 32° C. and 72° C., depending on primer length and nucleotide composition. In high stringency PCR amplification, a temperature at or slightly (up to 5° C.) above primer Tm is typical, although high stringency annealing temperatures can range from about 50° C. to about 72° C. and are often 72° C., depending on the primer and buffer conditions (Ashen et al, Clin. Chem. 47:1956-61 (2001)).

Suitable oligonucleotide primers for detection and amplification of ARNT isoform 3 polynucleotides typically ranges from about 10 to about 50 nucleotides, and include the three primer sets shown in Table 1.

| TABLE 1 | |
|---|---|
| Forward primer: | |
| 5′-ACTGCCAACCCCGAAATGAC-3′ | (SEQ ID NO: 1) |
| Reverse primer: | |
| 5′-CCGCCGTTCAATTTCACTGT-3′ | (SEQ ID NO: 2) |
| Forward primer: | |
| 5′-TGGAATTCAAGGTGGAGGAG-3′ | (SEQ ID NO: 3) |
| Reverse primer: | |
| 5′-TGTGATTTTCCCTGGCAAAC-3′ | (SEQ ID NO: 4) |
| Forward primer: | |
| 5′-ATGTACCATCACTGGGTCCA-3′ | (SEQ ID NO: 5) |
| Reverse primer: | |
| 5′-TGATGTAGGCTGTCATCTTG-3′ | (SEQ ID NO: 6) |

Methods of measuring protein levels in biological samples are also known in the art. Many such methods employ antibodies (e.g., monoclonal or polyclonal antibodies) that bind specifically to target proteins. In such assays, an antibody itself or a secondary antibody that binds to it can be detectably labeled. Alternatively, the antibody can be conjugated with biotin, and detectably-labeled avidin (a polypeptide that binds to biotin) can be used to detect the presence of the biotinylated antibody. Combinations of these approaches (including “sandwich” assays) familiar to those in the art can be used to enhance the sensitivity of the methodologies. Some of these protein-measuring assays (e.g., ELISA, Western blot, dot-blot, dip-stick) can be applied to bodily fluids or to cell lysates, and others (e.g., immunohistological methods or fluorescence flow cytometry) applied to unlysed tissues or cell suspensions. Methods of measuring the amount of a label depend on the nature of the label and are known in the art. Appropriate labels include, without limitation, radionuclides (e.g., ¹²⁵I, ¹³¹I, ³⁵S, ³H, and ³²P) for using in radioimmunoassays, enzymes (e.g., alkaline phosphatase, horseradish peroxidase, luciferase, and β-glactosidase), fluorescent moieties or proteins (e.g., fluorescein, rhodamine, phycoerythrin, GFP, and BFP) or luminescent moieties (e.g., Qdot™ nanoparticles; Quantum Dot Corporation, Palo Alto, Calif.) for use in fluoroimmunoassays. Other applicable assays include quantitative immunoprecipitation or complement fixation assays.

The antibodies that may be used in the methods include any antibody that specifically recognizes and binds a selected isoform of ARNT, such as ARNTiso1, ARNTiso2 or ARNTiso3. The term “antibody” includes polyclonal antibodies, monoclonal antibodies, chimeric antibodies, humanized antibodies, and single chain antibodies (such as Fab, F(ab′)₂, Fab′, Fv, dAbs and single chain antibodies (scFv) containing a V_{L} and V_{H} domain joined by a peptide linker. The scFv's may be covalently or non-covalently linked to form antibodies having two or more binding sites).

The antibodies can be prepared against an ANRT isoform, such as isoform 3, using the full-length polypeptide or a fragment thereof. For example, a short peptide sequence of consecutive amino acids that appears in ARNTiso3 but not in ARNT isoform 1 could be used as the antigen. Such peptides include: ERFARENHSE, KERFARENHSEI, and KERFARENHSEIE. Antibodies so produced can be purified on an affinity column consisting of the antigen peptide and then tested for specificity to the ARNT isoform by Western blot analysis or other means known in the art.

In accordance with a preferred embodiment of the invention, a sample of a body fluid or tissue is contacted with an antibody which binds specifically to ARNT isoform 3 to form a complex, the first antibody being immobilized on a solid support. Sufficient time is allowed to permit binding of the ARNT isoform of the sample to the immobilized antibody. The solid support is then washed and contacted with a second antibody which binds specifically to the first antibody and is labeled with a detectable label or has attached to it a signal-generating system. The label or generated signal bound to the solid support is determined, providing a measure of the complex present in the sample, and hence determining the level of ARNT isoform in the sample.

The present invention also includes kits for use in performing the methods of the invention. Such kits may include the following components: one, two, or more oligonucleotide primers for use in detecting ARNTiso3 in a biological sample and instructional material describing how to use of the primer(s) in determining the presence or absence ARNTiso3 in the sample. Other kits may include the following components: one or more antibodies for use in detecting ARNTiso3 in a biological sample, such as an antibody that specifically binds ARNTiso3, and instructional material describing how to use of the antibody in determining the presence or absence ARNTiso3 in the sample.

Aminoflavone acts as an arylhydrocarbon receptor (AhR) agonist. The mechanism of AF activation, as an agent for breast cancer therapy, has been proven to be through AF binding and activation of the AhR. Other AhR ligands also bind AhR and elicit an anti-cancer response in the same manner or through the same pathway as AF (Dohr, O., et al., Arch Biochem Biophys, 321:405-412 (1995); Loaiza-Perez, A. I., et al., Mol Cancer Ther, 3:715-725 (2004); Okino, S. T., et al., Cancer Prey Res (Phila Pa) 2:251-256 (2009); Zhang, S., et al., Endocr Relat Cancer 16:835-844 (2009)). The correlation between the expression of ARNTiso3 by cancer cells and susceptibility or sensitivity of the cancer cells to AF disclosed herein, as well as the evidence provided that demonstrates AF-activated AhR cross talk with the estrogen receptor, makes it clear that all AhR-activating ligands (AhR agonists) have the potential to be effective anti-cancer agents. AhR agonists include flavonoids, such as AF and derivatives thereof, and other compounds. As an example, 2,3,7,8-Tetrachlorodibenzo-p-dioxin (TCDD) is an AhR agonist can also act as an anti-estrogen with similar effects on the same AF breast cancer cell lines, yet is not an aminoflavone or its derivative (Zhang, S., et al., Endocr Relat Cancer, 16:835-844 (2009); Frericks, M., et al., Toxicol Appl Pharmacol, 232:268-279 (2008); Matthews, J., et al., Mol Cell Biol, 25:5317-5328 (2005); Wang, W. L., et al., Carcinogenesis, 18:925-933 (1997)). Additional AhR agonists include 7,12-dimethylbenz[a]anthracene (DMBA), indolo-(3,2-b)-carbazole, 3,3′-diindolylmethane, sulforaphane, resveratrol (3,4′,5-trihydroxy-trans-stilbene), leflunomide, flutamide, nimodipine, omeprazole, mexiletine, and atorvastatin. However, the AhR agonists to be used in the methods disclosed herein are only limited in that the cancer cells expressing ARNTiso3 must be sensitive or susceptible to an activity of the compound.

As used herein, “AF” and “aminoflavone” is 5-amino-2-(4-amino-3-fluorophenyl)-6,8-difluoro-7-methylchromen-4-one (NSC 686288). A “derivative” of AF is any one of the natural or synthetic prodrugs, analogs and derivatives of AF known to those of skill in the art. A preferred derivative of AF is the prodrug AFP-464 (NSC 710464). AFP-464 is a lysyl prodrug of AF and it is synthesized to improve the aqueous solubility of the parent compound. AFP-464 undergoes rapid conversion to AF in plasma by nonspecific plasma esterases. Other suitable derivatives include those disclosed in Akama, T., et al. (Novel 5-Aminoflavone Derivatives as Specific Antitumor Agents in Breast Cancer. J. Med. Chem., 39(18):3461-3469 (1996), as well as those disclosed in WO/1996/024592, published Aug. 15, 1996, and in U.S. Pat. No. 6,812,246. Previous studies have indicated that human tumor cell lines exhibit particular sensitivity to AF including those of breast and renal origin. Previous studies with human breast and renal cancer cell lines showed that AF induced CYP1A1/1A2 and CYP1B1 protein expression and was converted to metabolites that were covalently bound to DNA. This resulted in phosphorylation of p53 and apoptosis.

Reference to “ARNT” and “aryl hydrocarbon nuclear translocator” herein includes all mammalian versions of the protein and gene encoding the protein. In one aspect, ARNT is human ARNT. The nucleic acid and amino acid sequence of human ARNT isoform 1 may be found under NCBI Reference Sequence NM_{—}001668.3. The nucleic acid and amino acid sequence of human ARNT isoform 2 may be found under NCBI Reference Sequence NM_{—}178426.1. The nucleic acid and amino acid sequence of human ARNT isoform 3 may be found under NCBI Reference Sequence NM_{—}178427.2.

While the correlation between expression of ARNTiso3 and susceptibility of the cancer to an activity of an AhR agonist, such as AF or a derivative thereof, has been most fully established in breast cancer, the correlation has also been found in other cancers. Therefore, the methods of the present invention can be practiced in conjunction with any cancer in which the correlation is found, including, for example, breast cancer, renal cancer, colon cancer, leukemia, and non-small cell lung carcinoma.

As used herein, the term “subject” refers to an animal, such as a mammalian species, including a human.

As used herein, an “activity” of an AhR agonist, such as AF, refers to any biological activity that has been ascribed to an AhR agonist, such as AF or a derivative thereof. Such activities include, but are not limited to: activation of the arylhydrocarbon receptor, formation of covalent adducts with RNA and/or DNA, induction of oxidative damage to DNA, induction of DNA double stranded breaks, and induction of apoptosis in a cell contacted with the compound.

The skilled artisan will understand that a cancer is “susceptible” or “sensitive” to an activity of an AhR agonist, such as AF or a derivative thereof, if the cancer as a whole or individual cells thereof have a deleterious reaction upon contact by the compound. The deleterious reaction can simply harm the cancer or cell in some manner, such as inhibition of metastasis or vascularization, or an induction of a decrease in cell growth, motility, or proliferation, or the reaction can be lethal to the cancer or cell, resulting, e.g., in a reduction in the size or volume of the cancer, or in cell death, such as through the induction of apoptosis in a cell contacted by the compound. Susceptibility or sensitivity can be determined in comparison to a cancer or cell not contacted by the compound. Susceptibility or sensitivity is an increase of at least about 1, 2, 3, 4, 5, 6, 7, 8, 9, 10, 15, 20, 25, 30, or 35% or more, in a deleterious reaction in comparison to a cancer or cell not contacted by the compound.

As used herein, “effective” in the context of the treatment of a subject having cancer using an AhR agonist, such as AF or a derivative thereof, means that administration of the compound to the subject results in one or more of a decrease in a symptom of the cancer, a decrease in cancer cell growth, motility, or proliferation, a reduction in the size or volume of the cancer, and cancer cell death. Effectiveness can be determined in comparison to a subject having the same cancer to which the compound is not being administered. Effectiveness is an increase of at least about 1, 2, 3, 4, 5, 6, 7, 8, 9, 10, 15, 20, 25, 30, or 35% or more, in one of the noted factors in comparison to a subject having the same cancer to which the compound is not being administered.

### EXAMPLES

### Cell Lines and Maintenance

Human breast cancer cell lines MCF7, T47D, MDA-MB-231 were kindly provided by Dr. Angelika Burger (Karmanos Cancer Center, Detroit, Mich.). The cell lines Hs578t and MDA-MB-468 cells were obtained from the American Type Culture Collection (Manassas, Va.). Cell lines were maintained in RPMI 1640 (Invitrogen) supplemented with L-glutamine, 10% (V/V) heat-inactivated fetal bovine serum (Hyclone), and 1% antibiotic-antimycotic (Invitrogen). All cells were maintained at 37° C. in a humidified incubator in 5% CO₂.

### Drugs and Chemicals

Aminoflavone, AF, (Kyowa Hakko Kogyo) was obtained from the Developmental Therapeutics Program of the National Cancer Institute. A 10 mM stock solution of AF was prepared by dissolving AF in DMSO. For use in cell-based assays, the AF stock solution was dissolved in cell culture media to arrive at the necessary concentrations.

### 1. AF Sensitivity of Breast Cancer Cells

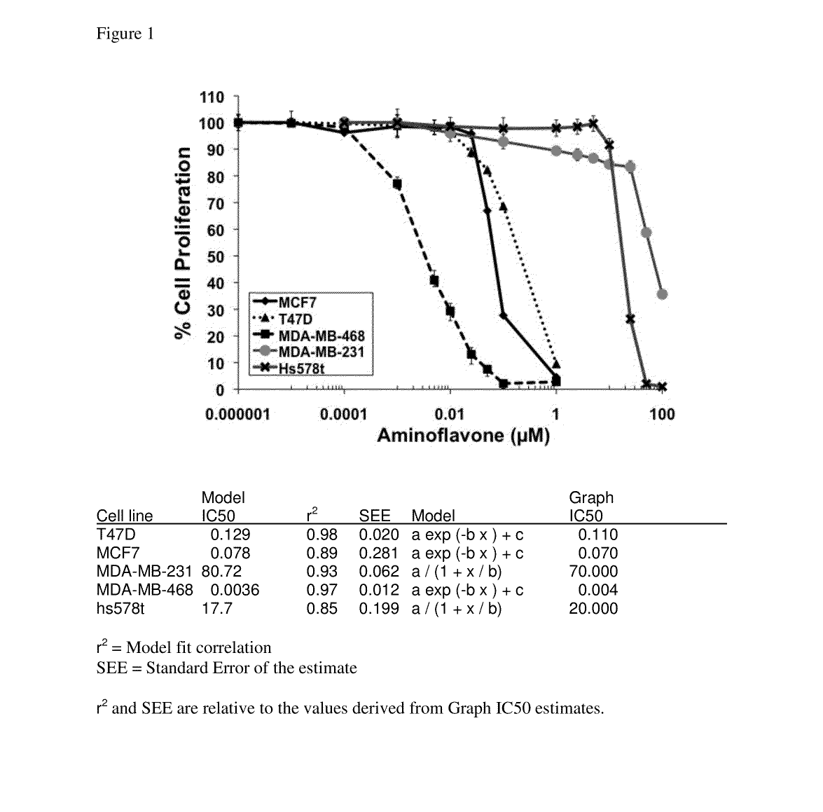

Concentration-effect assays were performed on breast cancer cell lines employing the MTS assay (Promega) to confirm previous studies which suggested that ER+ breast cancer cell lines were AF-sensitive, and that ER− cell lines were AF insensitive (FIG. 1).

The CellTiter 96 Aqueous MTS Reagent (Promega, Cat. G5421 WI) was employed to measure the effects of AF on cell proliferation. Briefly, cells in 100 μl were seeded in 96-well plates (Nunc) at a density of 2,000 cells/well and allowed to attach overnight. AF was added the following day in final concentrations of 0.1 nM to 100 μM in replicas of 8. Proliferation was measured 5 days later by adding 20 μl MTS reagent to the plates and incubation at 37° C. for 2 hours. Viable cells converted the MTS reagent to a formazan product, which was measured 4 hours later at 490 nm using a Synergy HT Multi-Detection Microplate Reader, and KC4 software (Bio-Tek). Cell proliferation was compared to DMSO treated controls as a percentage, against the background growth at the time of AF treatment.

For IC50 (50% Inhibiting Concentration) determination, vehicle treated cells were deemed as 100% cell growth. Percent growth inhibition was calculated by dividing AF treated MTS values by MTS values of vehicle treated cells, subtracting background (media alone without cells) from both prior to calculations. As such values do not represent true percent growth inhibition (GI) since the initial 2000 cells seeded were not subtracted from either AF treated or untreated cell lines. The data as calculated is more representative of percent cell survival and therefore defined as Inhibitory Concentrations (IC50) rather than GI50 values. That said, they provide for a highly accurate means for comparing effects of AF. IC50 values were estimated directly from the graph (FIG. 1), though model fitting employing BiodataFit 1.02 (Chang Biosciences, found on the world wide web at changbioscience.com/stat/ec50ht.html) provided for highly similar results with model fitting correlations of 0.85 or above and generally small SEE values (standard error of the IC50 estimate).

In agreement with the NCI study, all ER+ breast cancer cell lines tested were found to be AF sensitive. However, the data indicates that ERα status may not afford a complete correlation with AF sensitivity regarding ER− breast cancer cell lines since the ER− breast cancer cell line MDA-MB-468 was found to be AF sensitive as well. In FIG. 1, ER+ breast cancer cell lines MCF7 and T47D are shown to be AF sensitive (50% Inhibitory Concentration; IC50=70 nM and 110 nM, respectively) and ER− Hs578t and MDA-MB-231 cell lines are shown to be AF insensitive (IC50=20 μM and 70 μM, respectively). However, MDA-MB-468 deviates from the ER+/AF correlation, as it is ER− and AF sensitive (IC50=4 nM). For the purpose of consistency, AF sensitive or responsive cells lines are those cell lines with an IC50 below 1 μM.

### 2. AF Induces Apoptosis and DNA Double-Stranded Breaks in AF-Sensitive Breast Cancer Cell Lines

Because the relative activation of a downstream activator of AF activity (Cyp1A1) was not as pronounced with MDA-MBA-468 compared to ER+ cancer cell lines (data not shown), experiments were conducted to verify that MDA-MBA-468 sensitivity was associated with double stranded DNA breaks and apoptosis as it is for ER+ and AF sensitive cell lines (Kuffel, M. J., et al., Mol Pharmacol, 62(1):143-53 (2002)).

To determine the induction of apoptosis, the Cell Death Detection ELISA^{PLUS} Assay (Roche Cat. No. 11774425001), which determines the degree of cytoplasmic mono- and oligonucleosomes, was employed as described by the manufacturer. Briefly, breast cancer cells (5,000 cells for MCF-7; 2,000 cells for MDA-MB-231; 15,000 cells for T47D; 15,000 cells for MDA-MB-468; 5,000 cell for Hst578) were plated on 96 well plates and treated the next morning with or without or 1 uM AF for 24 h or 48 h. Cells were then concentrated by centrifugation at 200 g for 10 min at room temperature and the supernatant was discarded. Cells were lysed, and cytoplasmic fractions containing fragmented DNA were transferred to streptavidin-coated microtiter plates preincubated with a biotinylated monoclonal anti-histone antibody. The amount of fragmented nucleosomes bound to anti-histone antibody was evaluated by peroxidase-conjugated monoclonal antibody using ABTS (2,2-azino-di[3-ethylbenzthiazoline sulfonate-6-diammonium salt]) as a substrate, and read in a microplate reader at 405 nm. Non-treated cells were employed as controls.

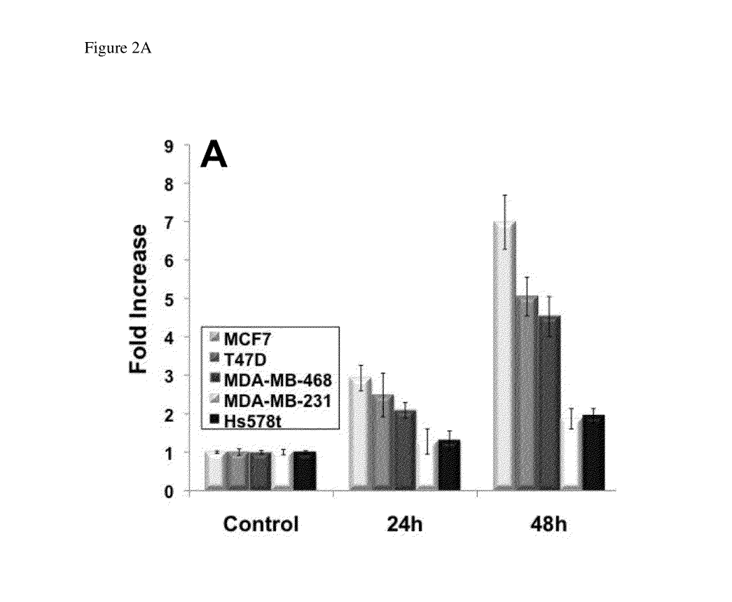

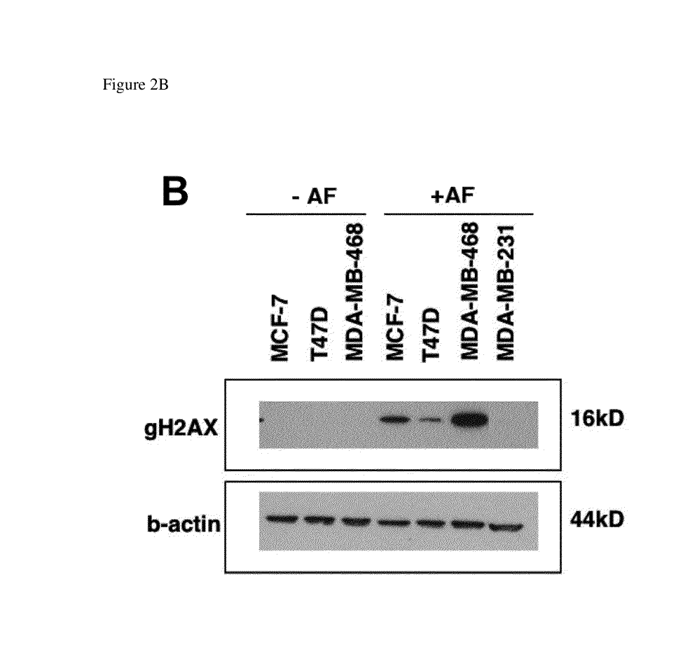

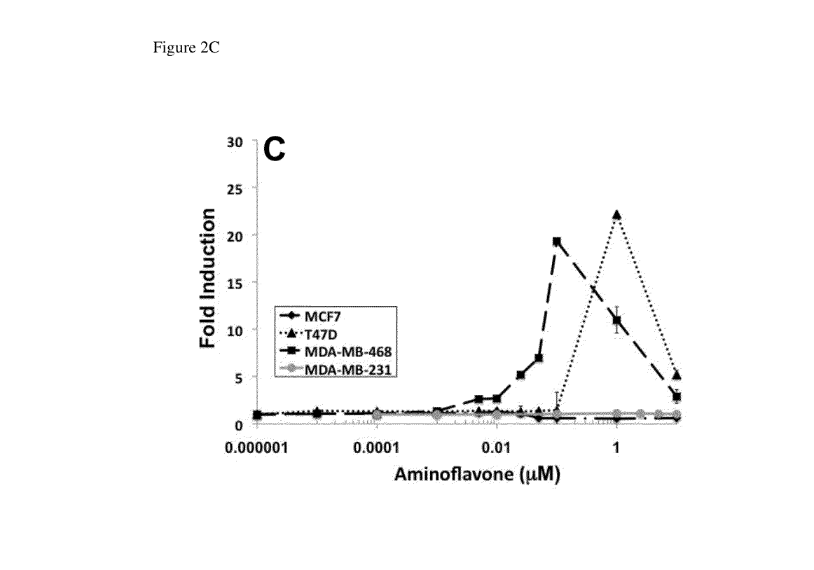

FIG. 2A indicates that at 1 μM AF, there exists a minimal increase in cytoplasmic nucleosomes for AF “insensitive” cell lines (MDA-MB-231 and Hst578t<2-fold, P<0.05) and a more substantial increase in inhibition of cell proliferation for AF sensitive cell lines MCF7, T47D and Hst578t (>4.5 fold). These higher levels of apoptosis are consistent with the presence of gamma-H2AX, and by inference double stranded DNA breaks, observed only for AF sensitive cells including MDA-MBA-468 (FIG. 2B). Gamma-H2AX was observed only after AF induction in AF sensitive cells and was not observed at all for the AF insensitive MDA-MBA-231 cell line.

Western blot analysis for phosphorylated H2AX (γ-H2AX) was performed by first growing cells to between 50% and 80% confluence and treating them with concentrations of AF for 24 hrs, according to the concentration at which 50% of the proliferation was inhibited (IC50). Cells were collected and centrifuged at 1,000×g for 15 minutes at 4° C. Histones were released by the method described by Meng et al. (Cancer Res, 65(12):5337-43 (2005)). Briefly, pellets were washed twice in PBS, homogenized in 0.2 mol/L H₂SO₄, and centrifuged at 13,000×g. The supernatant was removed and 0.25 volume of 100% (w/v) trichloroacetic acid was added to precipitate the histones. Samples were then centrifuged again at 13,000×g for 15 mins at 4° C. The supernatant was removed and the remaining pellet was suspended in 100% ethanol overnight. A final centrifugation step was performed at 13,000×g for 15 mins at 4° C. The solute was then dissolved in nuclease-free water. Protein concentration was determined using the Bio-Rad Protein Assay (Bio-Rad, CA). 30 μg of protein was resolved on 4-20% Tris-glycine precast gels (Invitrogen). Proteins were transferred onto a PVDF membrane (Immobilon-P, Millipore) and then blocked with 5% milk in TBST (0.1% Tween-20 in 1× Tris-buffered saline—pH 7.4) for 1 hr. Immunoblotting was performed by overnight incubation of mouse anti-γ-H2AX antibody (Upstate) at a dilution of 1:1000 in 5% milk in TBST, at 4° C. The blots were washed and then incubated with anti-mouse HRP antibody (Sigma) at a dilution of 1:5000 in 5% milk in TBST. Protein expression was visualized by chemiluminescence (Amersham Biosciences, PA). Mouse Anti-b Actin monoclonal antibody (Sigma) was used as control, according to the same method in order to ensure proper loading of the protein.

The precise apoptotic mechanisms AF induces may also involve cell-specific responses. For example, MDA-MBA-468 and T47D showed marked induction of caspases 3/7, in an assay detecting the combined activity of apoptotic caspases 3 and 7, whereas for MCF7 caspases 3/7 were not activated (FIG. 2C). Though MCF7 cells are deficient for caspase 3 activity they maintain functional caspase 7 (Kagawa, S., et al., Clin Cancer Res, 7(5):1474-80 (2001)).

The Caspase Glo-3/7 Assay (Promega) was used to measure the combined activities of caspases-3 and -7. In brief, cells were seeded in a white-walled 96-well plate (Nunc) at a density of 2,000 cells/well and allowed to attach overnight. AF was added the following day in final concentrations of 0.1 nM to 100 μM in replicas of 8. Caspase activity was measured 5 days later by adding the Caspase Glo-3/7 reagent to the plate and incubating for another 2 hours. Caspase cleavage results in the release a substrate for luciferase that was measured using a LumiCount luminometer (Packard). Caspase activity levels were normalized to the amount of viable cells, determined by the CellTiter-Glo Luminescent Cell Viability Assay (Promega) performed in parallel to the caspase assay. Data analysis to obtain the mean and standard error as well as graphing employed Excel® (Microsoft Corporation).

### 3. AhR and ER Crosstalk

Considering that the AhR and ER are both nuclear receptors and act as transcription factors, modulation of ER action after AhR activation by AF (“crosstalk”) was theorized to contribute to overall AF effects in ER+ cell lines. Therefore, Chromatin Immunoprecipitation (ChIP) was employed to study the potential crosstalk of AhR and ER after AF action at the PS2 promoter, a classical ER/estradiol inducible gene, and on Cyp1A1, a classical AF/AhR inducible gene known to be induced by AF. Transcription components studied were the AhR, ER, ARNT (the AhR transcriptional partner), RNA Polymerase II and CBP. CBP is a histone acetyl transferase associated with productive AhR induction of gene expression (Hestermann, E. V. and M. Brown, Mol Cell Biol, 23(21):7920-5 (2003)). In both T47D and MCF7 ER+ cell lines, all transcription components tested were present on the Estradiol inducible PS2 gene promotor, including the AhR. On the Cyp1A1 promotor, all the apparatus excluding CBP was present.

ChIP analysis was performed employing the Chromatin Immunoprecipitation (ChIP) Assay Kit (Millipore) as per company recommendations. Briefly, MCF-7 or T47D cells were grown in 100-mm dishes to 70-80% confluency without or with 1 uM AF for 4 hr, and 8 hrs. Cells were cross-linked with 1% formaldehyde, harvested, hypotonically lysed, and nuclei were collected. Nuclei were sonicated to shear DNA to lengths between 200 to 500 bp as observed from agarose gel electrophoresis (not shown). The chromatin was then pre-cleared by protein-A agarose/Salmon Sperm DNA. These “input” samples represent total DNA processed, and a sample of each was saved as PCR control. Pre-cleared input samples were then incubated with IgG antibodies specific to Actin, (sc-8432, Santa Cruz Biotechnology), as normal control, ERα: (sc-543X, Santa Cruz Biotechnology), AhR (sc-5579X, Santa Cruz Biotechnology), Arnt (sc-5580X, Santa Cruz Biotechnology) or RNA Polymerase II (sc-56767, Santa Cruz Biotechnology) at 4° C. overnight. Samples were then precipitated by the addition of Protein G plus/Protein A beads for 1 hour, with extensive washing of the beads. The protein-DNA cross-links were eluted and reversed as recommended by the manufacturer. DNA was recovered by phenol/chloroform and ethanol precipitation.

The resultant DNA was analyzed by PCR employing GoTaq Green Master Mix (Promega) using the following protocol: 94° C. for 2 minutes for initial melting, followed by 35 cycles at 94° C. for 30 seconds, 55° C. for 40 seconds, 72° C. 2 for minutes, and followed by a single extension at 72° C. for 10 minutes. Primers for amplification of promoter regions were CYP1A1, 5′-ACCCGCCACCCTTCGACAGTTCC-3′ (SEQ ID NO:7) and 5′-CTCCCGGGGTGGCTAGTGCTTTGA-3′ (SEQ ID NO:8) which amplifies a 397 bp region of the CYP1A1 promoter, for the pS2 promotor: 5′-GATTACAGGCGTGAGCCACT-3′ (SEQ ID NO:9), and 5′-CTCCCGCCAGGGTAAATACT-3′ (SEQ ID NO:10) amplifying a 233 bp fragment and negative control primers 5′-ATGGTTGCCACTGGGGATCT-3′ (SEQ ID NO:11) and 5′-TGCCAAAGCCTAGGGGAAGA-3′ (SEQ ID NO:12), which amplifies a 174-bp fragment genomic DNA between the GAPDH gene and the CNAP1 gene (Higgins, K. J., et al., Mol Endocrinol, 22(2):388-402 (2008)).

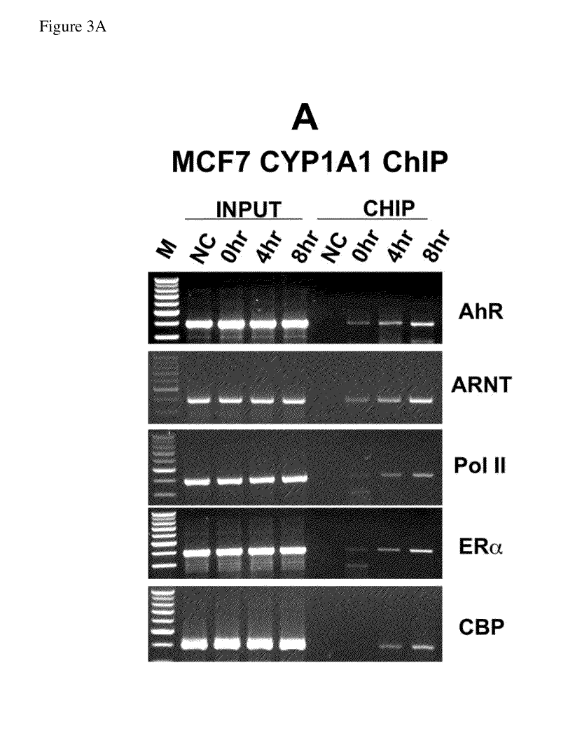

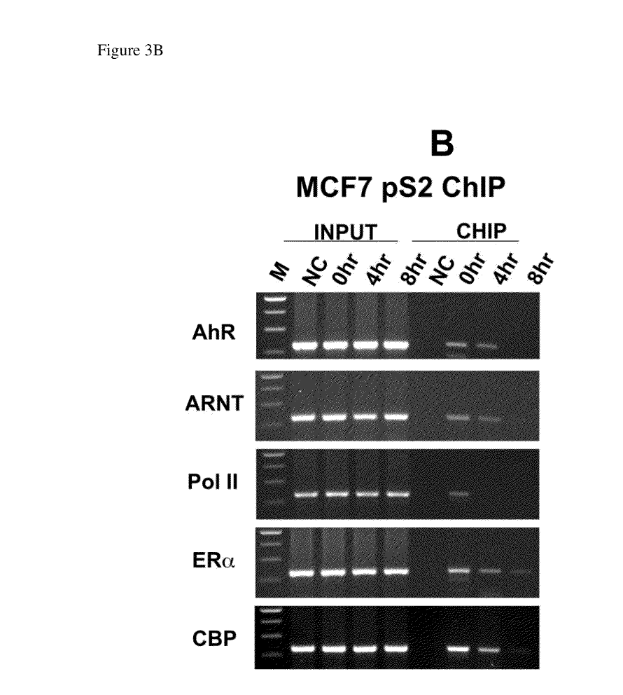

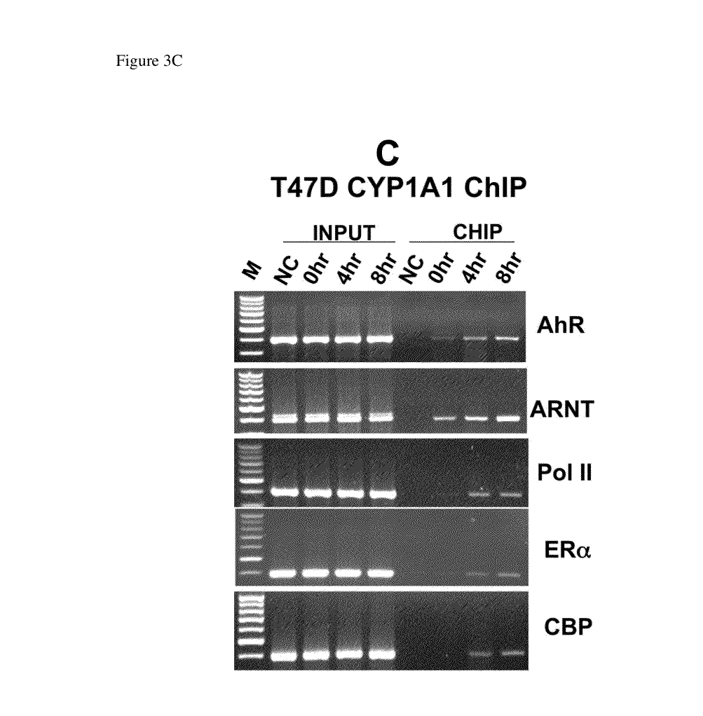

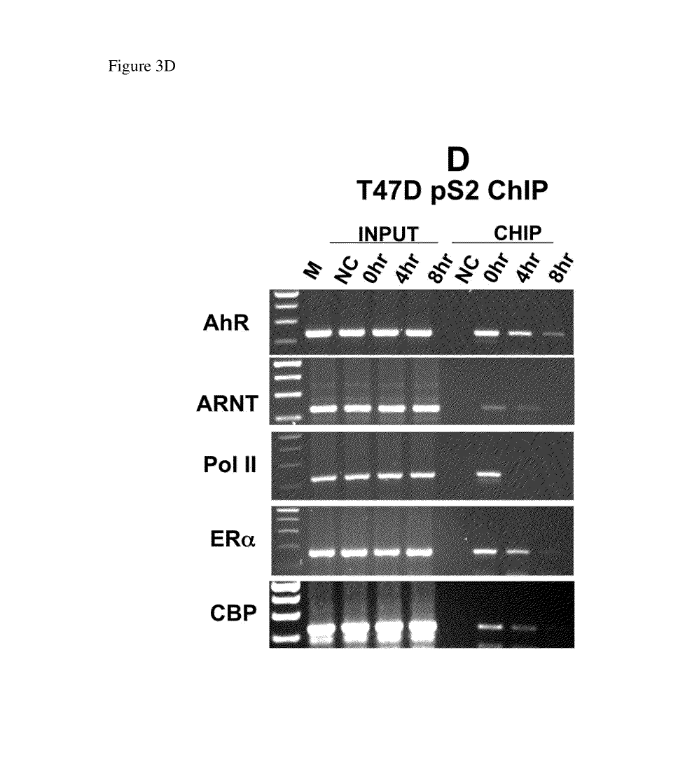

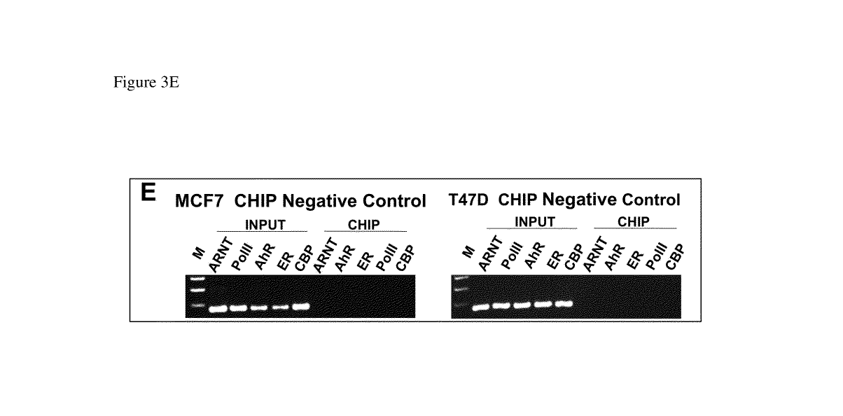

Upon addition of AF a clear and striking pattern of transcriptional adjustment occurred for both T47D and MCF7 cell lines. AF induced the dissociation of ER-related transcriptional control elements from the PS2 estrogenic promoter and their association with the Cyp1A1 promotor (FIG. 3). A very telling finding is the total dissociation of RNA Polymerase II from the pS2 gene, since without RNA Polymerase II, transcription cannot proceed. It is also worth noting that CBP may play a key role in CYP1A1 gene activation since of all the components studied CBP alone was not detected on the CYP1A1 gene until AF induction. Concomitant with AF induced CBP binding to the CYP1A1 promotor, CBP dissociated from the PS2 gene. Together, the data strongly suggests that AF can activate an inverse transcriptional crosstalk between the ER and the AhR.

4. ARNT Isoform 3 (ARNTiso3) is Associated with AF Sensitivity

As demonstrated MDA-MB-468 cells, ER status may not completely correlate with AF sensitivity. Thus, a biomarker independent of ER was sought for use in determining which breast cancer cells might be AF sensitive. Assuming an AhR-ER transcriptional crosstalk, elements of the transcription machinery represented a reasonable path toward such biomarkers. Specific AhR single nucleotide polymorphisms that may play a role in sensitivity to ligands were tested, including G1661A and T3801C, though no correlation to AF sensitivity was found (data not shown) (Cauchi, S., et al., Carcinogenesis, 22(11):1819-24 (2001); Chen, D., et al., Pharmacogenet Genomics, 19(1):25-34 (2009)).

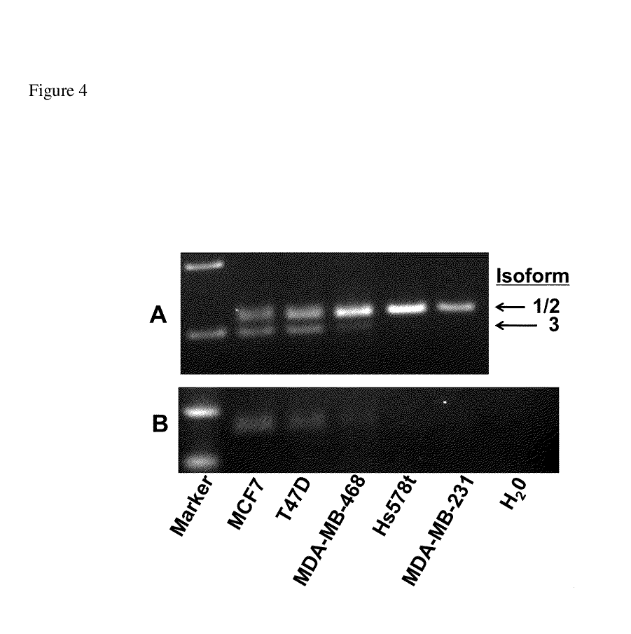

With respect to ARNT polymorphisms, there exist three ARNT mRNA isoforms: ARNTisol (NM_{—}001668.3), ARNTiso2 (NM_{—}178426.1) and ARNTiso3 (NM_{—}178427.2). Both isoforms 1 and 2 contain exon 5, a 45 base exon, which is lacking in ARNTiso3. Isoform 1 encodes the longest transcript. Relative to isoform 1, isoform 2 is a truncated isoform as it lacks several exons and it also contains a distinct C-terminus. Employing RT-PCR with PCR primers that lie outside of the 45-base exon 5, ARNT isoforms 1 and 2 were detected as a single PCR product, and a smaller band corresponding to ARNTiso3 was detected, when present (FIG. 4A). To verify these findings, in an RT-PCR “single band assay”, one of the primers included sequences unique to the ARNTiso3 splice site, providing for a single ARNTiso3 fragment (FIG. 4B).

The signal from ARNTiso3 in the single band assay was not very robust even after 30 PCR cycles and as such the noted primers were noted used for quantitative PCR. Regardless, it was found that the presence of ARNTiso3, as detected within the parameters of the assay, fully correlated with AF sensitivity in breast cancer cell lines. Cells insensitive to AF, such as MDA-MB-231, lacked ARNTiso3 and cell lines presenting ARNTiso3, including the ER− cell line MDA-MB-468, were all AF sensitive.

To detect the ARNTiso3, total RNA was extracted from breast cancer cell lines employing the RNAeasy® Plant Mini Kit (Qiagen) following the manufacturer's directions (1×10⁶ cells were harvested and total RNA were purified following the directions.). RNA extracts of the NCI 60-cell line panel was kindly provided by the NCI DTP program. 3 μg RNA was converted to cDNA by reverse transcription with M-MLV Reverse Transcriptase (Invitrogen) employing 50 ng random primer NNNNNN (where N represent a randomized base) and 50 ng of 16 base long Oligo dT. Samples were heated to 70° C. for 10 min and chilled on ice, followed by the addition of 5× buffer, Dithiothreitol (DTT), dNTP and M-MLV Reverse Transcriptase as suggested by the manufacturer. Samples were then incubated at 25° C. for 10 minutes, 37° C. for 60 minutes and inactivate by heating at 70° C. for 15 min. PCR amplification of ARNT fragments employed the forward primer 5′-ACTGCCAACCCCGAAATGAC-3′ (SEQ ID NO:1), and the reverse primer 5′CCGCCGTTCAATTTCACTGT-3′ (SEQ ID NO:2), producing a 288 (ARNTiso1 or 2) or a 243 base pair fragment (ARNTiso3). For validation, a second PCR assay employed the forward primer 5′-TGGAATTCAAGGTGGAGGAG-3′ (SEQ ID NO:3) and the reverse primer, 5′-TGTGATTTTCCCTGGCAAAC-3′ (SEQ ID NO:4) generating a single 155 base pair product, when present. The reverse primer overlaps the “absent” exon 5 ARNT isoforms 1 and 2 so that the reverse primer is unique for ARNTiso3 alone. Amplified PCR fragments were separated employing 3% NuSieve® 3-1 Agarose gel electrophoresis (Lonza Rockland, Inc.) and staining with ethidium bromide.

### 5. ARNTiso3 is Predictive of AF Sensitivity in Cancer of Breast Origin

Considering potential clinical implications of a biomarker for AF sensitivity, a correlation between ARNTiso3 and AF sensitivity in cancer cell lines other than those of breast origin was tested. To this aim, the NCI DTP program kindly provided RNA samples derived from 60-cell lines that were used in their small molecule screen for cancer inhibitors (Holbeck, S. L., Eur J Cancer, 40(6):785-93 (2004)). Data for AF sensitivity (and other compounds) in the 60-cell line panel determined by the NIH is available online on their website (dtp.nci.nih.gov/docs/dtp_search.html).

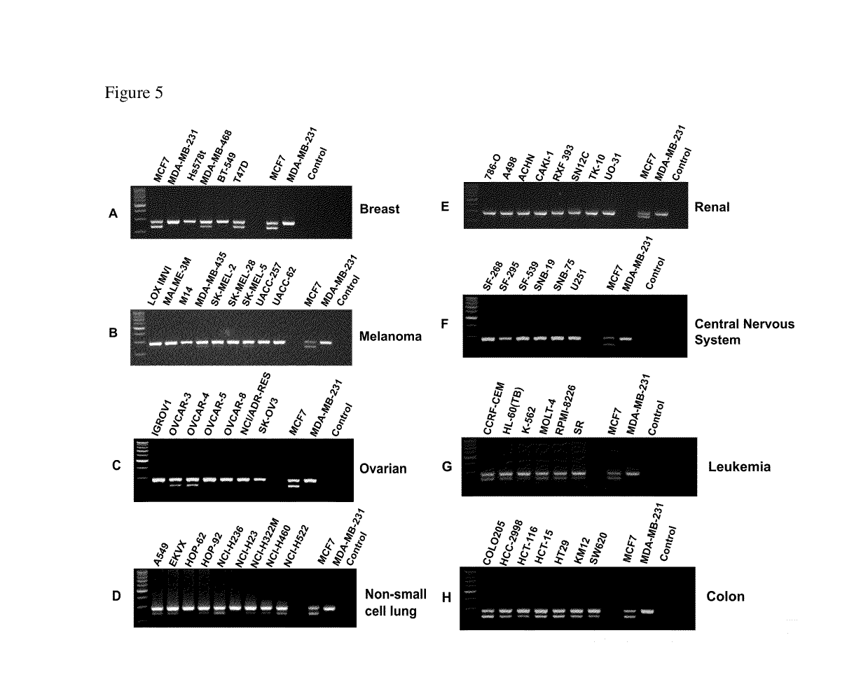

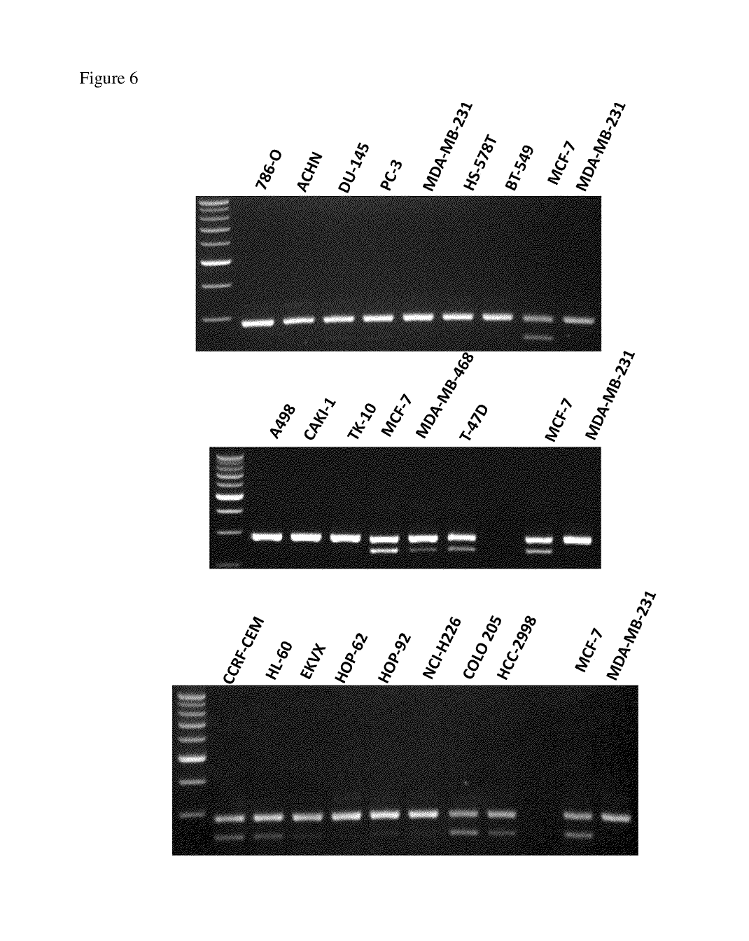

RT-PCR of the 60 cell lines was performed with the same primers employed in FIG. 4. In addition, twenty-four of the sixty cell lines were “spot-checked” with a PCR reaction using alternate primers surrounding exon 5, providing for an additional verification (FIG. 6). In FIG. 5, cells of similar origin were PCR assayed as distinct panels with MCF7 and MDA-MB-231 cell line RNA employed as controls (FIGS. 5A-H).

FIG. 5A contained the breast cancer cell lines within the 60-cancer cell panel. The presence/absence of ARNTiso3 in FIG. 5A concurs with ARNTiso3 as correlated with the AF-sensitive breast cancer cell line data generated in the inventors' laboratory. A breast cancer cell line present in the panel but not studied for AF sensitivity is BT-549, negative for ARNTiso3 in the assay and AF in-sensitive as determined by the NIH study, further supporting the AF-ARNTiso3 correlation.

For the NIH 60-cancer cell line panel, results of RT-PCR of ARNTiso3 herein, and AF dose response data from the NIH studies, indicate that a correlation primarily between the two for breast cancer cell lines. Renal cancer cell lines all lack ARNTiso3 yet some renal cell lines, namely A496, CAKi1 and TK10 are AF sensitive (FIG. 5E). Central nervous system (CNS) cell lines also lack ARNTiso3 and are AF insensitive, though some degree of sensitivity was observed for CNS cancer cell line U251 (FIG. 5F). Leukemia and colon cancer cell lines (FIGS. 5G and H) all present ARNTiso3, however only colon SW620 and K562 leukemia cell lines are AF sensitive. Selected detection of ARNTiso3 exists in the Melanoma and Ovarian panels yet these do not correlate with AF sensitivity. There is also no correlation between AF and ARNTiso3 in the non-small cell lung carcinoma panel, where all cell lines presented either a faint or stronger ARNTiso3 band, yet only select cell lines are sensitive.

All documents, books, manuals, papers, patents, published patent applications, guides, abstracts and other reference materials cited herein are incorporated by reference in their entirety and to the same extent as if each independent publication or patent application was specifically and individually indicated to be incorporated by reference.

While the invention has been described in connection with specific examples and embodiments thereof, it will be understood that it is capable of further modifications and this application is intended to cover any variations, uses, or adaptations of the invention following, in general, the principles of the invention and including such departures from the present disclosure come within known or customary practice within the art to which the invention pertains and may be applied to the essential features hereinbefore set forth, and follows in the scope of the appended claims.

1. A method for determining whether a selected cancer is susceptible to an activity of an AhR agonist, comprising screening the cancer for expression of an isoform of aryl hydrocarbon nuclear translocator (ARNT).

2. The method of claim L wherein the isoform of ARNT is isoform 3 of ARNT.

3. A method for determining whether treatment of a subject having cancer with an AhR agonist will be effective, comprising screening the cancer for expression of an isoform of ARNT.

4. The method claim 3, wherein the isoform of ARNT is isoform 3 of ARNT.

5. A method for screening a subject having cancer for sensitivity to treatment with an AhR agonist, comprising assaying a biological sample obtained from the subject for expression of an isoform of ARNT.

6. The method of claim 5, wherein the isoform of ARNT is isoform 3 of ARNT.

7. The method of claim 1, wherein the screening is via polymerase chain reaction (PCR) or an immunoassay.

8. The method of claim 5, wherein the assaying is via PCR or an immunoassay.

9. The method of claim 3, wherein the determining is conducted before the subject begins treatment.

10. The method of claim 5, wherein the screening is conducted before the subject begins treatment.

11. The method of claim 1, wherein the AhR agonist is aminoflavone (AF) or a derivative thereof.

12. The method of claim 1, wherein the cancer is breast cancer.

13. The method of claim 5, wherein the biological sample is a tissue biopsy.

14. The method of claim 11, wherein AF or a derivative thereof is AFP-464.

15. The method of claim 7, wherein the immunoassay is performed using an anti-ARNTiso3 specific antibody.

16. The method of claim 8, wherein the immunoassay is performed using an anti-ARNTiso3 specific antibody.

17. The method of claim 3, wherein the screening is via polymerase chain reaction (PCR) or an immunoassay.

18. The method of claim 3, wherein the AhR agonist is aminoflavone (AF) or a derivative thereof.

19. The method of claim 5, wherein the AhR agonist is aminoflavone (AF) or a derivative thereof.

20. The method of claim 3, wherein the cancer is breast cancer.

21. The method of claim 5, wherein the cancer is breast cancer.

22. The method of claim 18, wherein AF or a derivative thereof is AFP-464.

23. The method of claim 19, wherein AF or a derivative thereof is AFP-464.

24. The method of claim 17, wherein the immunoassay is performed using an anti-ARNTiso3 specific antibody.


SEQUENCE LISTING (12 sequences)

SEQ ID NO: 1 - DNA, 20 bp, Artificial Sequence
  chemically synthesized oligonucleotide primer
actgccaaccccgaaatgac

SEQ ID NO: 2 - DNA, 20 bp, Artificial Sequence
  chemically synthesized oligonucleotide primer
ccgccgttcaatttcactgt

SEQ ID NO: 3 - DNA, 20 bp, Artificial Sequence
  chemically synthesized oligonucleotide primer
tggaattcaaggtggaggag

SEQ ID NO: 4 - DNA, 20 bp, Artificial Sequence
  chemically synthesized oligonucleotide primer
tgtgattttccctggcaaac

SEQ ID NO: 5 - DNA, 20 bp, Artificial Sequence
  chemically synthesized oligonucleotide primer
atgtaccatcactgggtcca

SEQ ID NO: 6 - DNA, 20 bp, Artificial Sequence
  chemically synthesized oligonucleotide primer
tgatgtaggctgtcatcttg

SEQ ID NO: 7 - DNA, 23 bp, Artificial Sequence
  chemically synthesized oligonucleotide primer
acccgccacccttcgacagttcc

SEQ ID NO: 8 - DNA, 24 bp, Artificial Sequence
  chemically synthesized oligonucleotide primer
ctcccggggtggctagtgctttga

SEQ ID NO: 9 - DNA, 20 bp, Artificial Sequence
  chemically synthesized oligonucleotide primer
gattacaggcgtgagccact

SEQ ID NO: 10 - DNA, 20 bp, Artificial Sequence
  chemically synthesized oligonucleotide primer
ctcccgccagggtaaatact

SEQ ID NO: 11 - DNA, 20 bp, Artificial Sequence
  chemically synthesized oligonucleotide primer
atggttgccactggggatct

SEQ ID NO: 12 - DNA, 20 bp, Artificial Sequence
  chemically synthesized oligonucleotide primer
tgccaaagcctaggggaaga